(19) United States
(12) Patent Application Publication
(10) Pub. No.: US 2023/0101581 A1
(43) Pub. Date: Mar. 30, 2023

(54) METHOD AND APPARATUS FOR PLANNING NAVIGATION

(71) Applicant: Siemens Healthcare GmbH, Erlangen (DE)

(72) Inventors: Christian Kaethner, Forchheim (DE); Michael Wiets, Langensendelbach (DE)

(21) Appl. No.: 17/953,320

(22) Filed: Sep. 26, 2022

(30) Foreign Application Priority Data
Sep. 27, 2021 (DE) 10 2021 210 757.5

(51) Int. Cl. A61B 34/10; A61B 34/20

(52) U.S. Cl. CPC A61B 34/10; A61B 34/20; A61B 2034/107; A61B 2034/301

(57) ABSTRACT

A method for planning a remote-controlled navigation of medical objects in a hollow organ of a patient. The navigation is performable by robot or in a robot-supported manner using a robot system, and is visually monitored by an imaging system. The robot system includes a drive system, a robot control unit, and at least one input unit arranged at a distance from the robot control unit. At least one data transmission link is present. The method includes supplying data for a planned navigation procedure of an object through a hollow organ with at least one navigation step, evaluating, by an evaluation system, the supplied data in terms of a performability level of the navigation procedure. The evaluation is carried out based on a comparison with empirical data and/or based on a theoretical model, and/or using a learning-based algorithm. The method includes outputting an evaluation result to an output unit.

This application claims the benefit of German Patent Application No. DE 10 2021 210 757.5, filed on Sep. 27, 2021, which is hereby incorporated by reference in its entirety.

### BACKGROUND

The present embodiments relate to planning a remote-controlled navigation of medical objects in a hollow organ of a patient according.

Invasive medical procedures in or via the vascular system of the human body use medical objects (e.g., devices, instruments, or guide wires) that are manually introduced into the vascular system and guided to the target region for treatment. Ordinarily, at least one imaging method (e.g., an X-ray imaging system) is employed as a supporting measure, enabling the person providing treatment to have real time monitoring and understanding, based on image data, of the progress of treatment (e.g., the position of the object). For many procedures, there is additionally a need, aside from the live recordings of image data during an operation, to refer back to pre-operative image data and also bring the pre-operative image data into the operation.

Traditionally, a person giving treatment, frequently supported by an assistant, stands directly at the positioning table of the patient to carry out a (e.g., planned) procedure. A further development of this medical method switches a robot system between the hands of the person giving treatment and the patient with the advantage that the person giving treatment no longer needs to stand directly at the patient's positioning table but may perform the maneuvering of the objects (e.g., rotational, forward, and backward movement) in a remote controlled manner. Fundamentally, robot systems of this type, by which a (semi-)automatic movement of an object (e.g., a catheter and/or guide wire) in an organ cavity of a patient may be brought about in a robot-supported manner, are known (e.g., from EP 3406291 B1). To do this, a corresponding user interface for the remote controlled movements is made available to the person giving treatment. Additionally, for the necessary visual feedback, X-ray images from the imaging device may be recorded, transmitted, and displayed to the person giving treatment. The advantage of this robot guidance of the medical object lies partly in the convenient working position of the person giving treatment, the option of being able to completely leave the irradiated zone at the patient table, and therefore the greater working safety due to the avoidance of radiation.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method that provides that a remote controlled, robot-supported navigation procedure visually monitored by an imaging system is particularly safe for a patient is provided. As another example, an X-ray device that is suitable for carrying out the method is provided.

The method for planning a remote-controlled navigation of medical objects in a hollow organ of a patient is provided. The navigation is capable of being performed by robot or in a robot-supported manner using a robot system and being visually monitored by an imaging system. The robot system has a drive system, a robot control unit, and at least one input unit arranged at a distance from the robot control unit. At least one data transmission link is present (e.g., between the robot control unit and the input unit). The method includes supplying data for a planned navigation procedure of an object through a hollow organ with at least one navigation step. The supplied data is evaluated by an evaluation system in terms of a performability level of the navigation procedure. The evaluation is carried out based on a comparison with empirical data and/or based on a theoretical model, and/or using a learning-based algorithm. An evaluation result is output to an output unit. In this way, a person giving treatment in invasive operations involving a navigation may monitor all relevant acts of the navigation in a simple manner. The person giving treatment may thus identify from the evaluation result whether and at which step problems may arise that adversely affect the performability of the procedure and where to relevantly intervene, alter, or postpone the operation. This is also important in the case of operations that are undertaken by remote access and with the use of a robot system that carries out various aspects of a procedure (semi-)automatically. Overall, the quality of an operation is enhanced, and safety is substantially improved by the method.

The navigation procedure may involve, for example, a previously planned navigation of the medical object in the hollow organ with one or more navigation steps. The planning may have been done, for example, using a known planning tool or may be retrieved from a memory.

The performability level represents a value for the absolute or relative performability of the planned navigation procedure. In this regard, various options may be provided. Thus, the performability level may contain a broad subdivision (e.g., performable/not performable), a subdivision into multiple stages, or a very precise indication (e.g., probability in percent). Additionally, the performability level may be dependent on the corresponding person giving treatment or the equipment used.

A comparison with empirical data, for example, may refer back to a memory or a table with a large quantity of previously performed and/or collected data, with which the current data is compared. The data may then be dependent on or independent of the person giving treatment, for example. Alternatively or additionally, one or more theoretical model calculations may be carried out to obtain a performability level. A learning-based algorithm (e.g., previously trained with a large quantity of previously performed and/or collected data) may also be used for the determination of the performability level. A dependence on the person giving treatment is also possible.

According to an embodiment, the output contains a probability with which the navigation procedure is performable. In this way, the person giving treatment may identify rapidly and by simple means whether performability of the procedure is reliably provided and may initiate corresponding acts.

Further, optical, acoustic, or haptic warnings may also be output if, for example, the evaluation produces the result that performability of the navigation procedure is not provided or at least is provided with low or medium probability.

According to a further embodiment, the data contains at least one property of the data transmission link to be used (e.g., a data transmission rate, such as the bandwidth), and the evaluation takes account of at least the property of the data transmission link. This is very helpful, for example, for a person giving treatment who is connected by remote input unit (e.g., arranged at a distance from the robot control unit), for checking the reliability of the treatment and to provide and therefore enhance patient safety.

For the most precise possible analysis of performability, according to a further embodiment, the data has a type of the planned navigation procedure, and/or a sequence of steps in the navigation procedure, and/or patient data (e.g., weight, and/or age, and/or height), and/or data for the hollow organ (e.g., structure and/or anatomy of the hollow organ), and/or object data, and/or a medicament to be applied, and/or a contrast agent to be used, and/or device data, and/or X-ray parameters, and/or user-specific data (e.g., which user is performing the treatment, etc.). In this way, the monitoring of the navigation procedure may be carried out on multiple or even all details, which further raises the reliability and therefore patient. Further, other variables that may be included in the analysis may also be provided.

According to a further embodiment, at least one suggestion for modifying the planned navigation procedure is output. The navigation procedure adapted by the modification has a higher or at least the same probability of performability. In this way, a person giving treatment is given a suggestion for an alternative to the procedure already planned, which in the best case may be performed more reliably, and may then make a decision about the implementation. The person giving treatment does not need to give detailed consideration himself as to how to modify the procedure but instead is rapidly given a suitable suggestion. The person giving treatment may then choose or reject the suggestion, for example.

For example, multiple suggestions containing navigation procedures differing from the planned navigation procedure may also be output with respective performability levels so that the person giving treatment may compare the multiple suggestions with each other and choose the one appropriate for the person giving treatment. The person giving treatment may then choose from the available suggestions, for example, or also reject the suggestions. This increases the flexibility of an operation.

In one embodiment, the at least one modification suggestion encompasses modifications with regard to at least one navigation step, with regard to the object used, with regard to the navigation path, with regard to the contrast agent, with regard to the medicament, and/or with regard to the X-ray parameters.

To enable use of the results for future navigation procedures, the evaluation and/or the evaluation result is appropriately deposited in a database or table (e.g., a look-up table) for use in a subsequent method.

According to a further embodiment, a starting signal for a navigation procedure is automatically triggered when the performability level reaches or exceeds a preset threshold value. Thus, navigation procedures identified as being capable of being performed particularly reliably may be started directly. The threshold value may lie at using a probability of 90%, 95%, or even 100%, for example. The threshold value may be selected and set in advance by a person giving treatment, for example.

The present embodiments also include an overall system for carrying out a method as described above, having a robot system with at least one robot control unit, a robot-supported drive system, and an input unit arranged at a distance from the robot control unit. The robot control unit is configured for activating a robot-supported navigation of a medical object in a hollow organ of a patient by the drive system. At least one data transmission link is present between the robot control unit and the input unit. The system includes an imaging system (e.g., an X-ray system) for visual monitoring of the navigation, with a beam source and an image detector for recording projection images. The system also includes a system control unit for activating the imaging system, an evaluation unit for evaluating supplied navigation planning data with regard to a performability level of a navigation procedure, and an output unit configured for outputting an evaluation result. The evaluation unit is configured to carry out the evaluation based on a comparison with empirical data and/or based on a theoretical model and/or by using a learning-based method. In one embodiment, the supplied data contains at least one property of the data transmission link, and at least the property of the data transmission link is taken into account in the evaluation. Additionally, at least a second data transmission link may also be present between the robot system and the imaging system, and the property of the second data transmission link may also be taken into account in the evaluation.

The present embodiments are explained in detail below based on schematically represented exemplary embodiments in the drawing, without this resulting in a limitation of the invention to these exemplary embodiments.

### BRIEF DESCRIPTION OF THE DRAWINGS

FIG. 1 shows acts of a method according to the present embodiments;

FIG. 2 shows further acts in a method according to the present embodiments;

FIG. 3 shows further acts in a method according to the present embodiments; and

FIG. 4 shows an embodiment of an overall system for carrying out the method.

### DETAILED DESCRIPTION

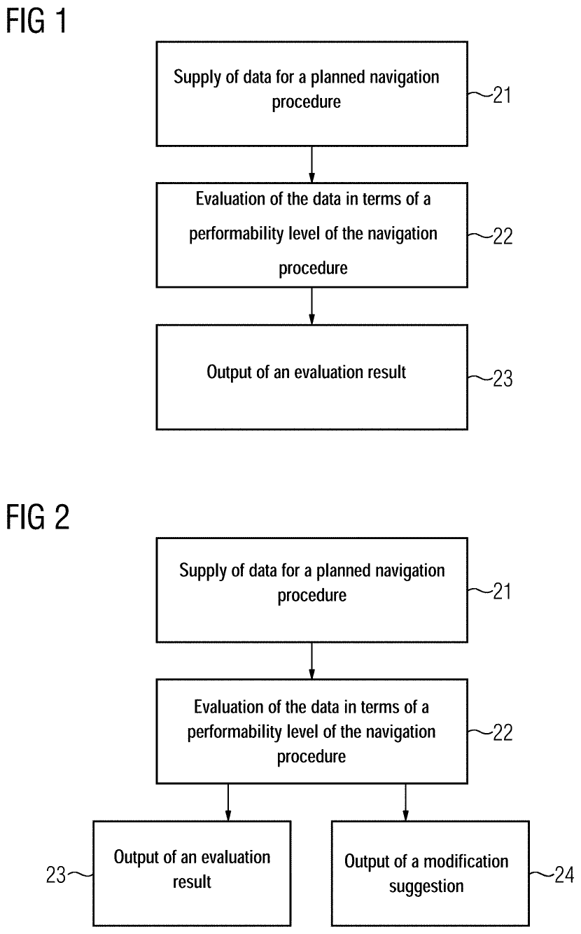

FIGS. 1 to 3 show method acts in a method for planning a remote-controlled navigation of medical objects in a hollow organ of a patient. The navigation is capable of being performed by robot or in a robot-supported manner using a robot system and is visually monitored using an imaging system, according to the present embodiments. Fundamentally, robot systems by which an automatic movement of an object in a hollow organ of a patient may be brought about in a robot-supported manner are known (e.g., from EP 3406291 B1).

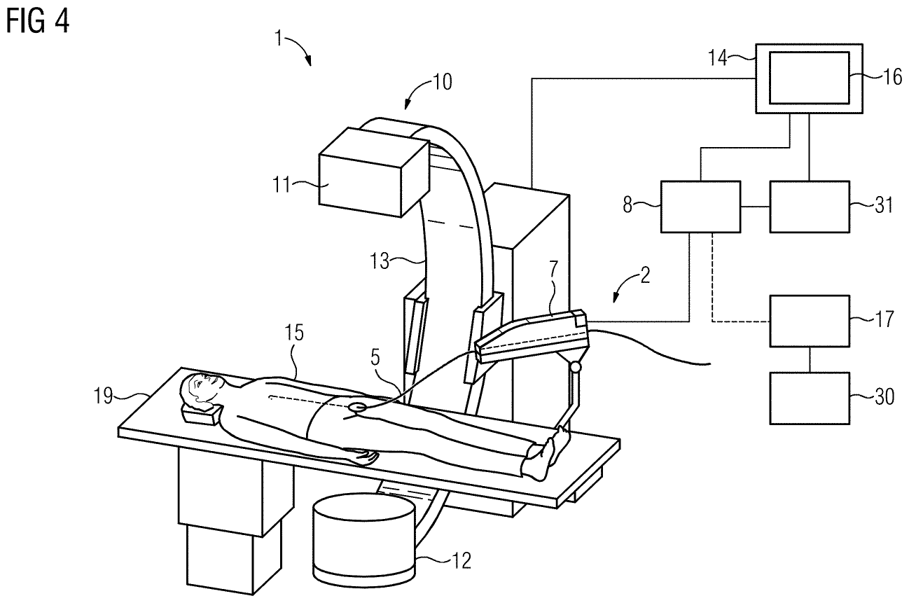

The overall system 1 for carrying out the method (shown in FIG. 4) has an imaging system in the form of an X-ray system 10 for recording X-ray images and a robot system 2. The X-ray system 10 may be configured, for example, from a C-arm X-ray device that is configured to be mobile or permanently installed. The X-ray system 10 has a C-arm 13, on which an X-ray source 12 and an X-ray detector 11 are arranged. A control unit 14 is provided for activation (e.g., a computer unit with a processor). Additionally, an evaluation unit 16 is present for evaluation of the data and information. The robot system 2 has at least one robot control unit 8 and a robot-supported drive system 7. The robot control unit 8 is configured to generate an activation signal for activating a robot-supported navigation of a medical object in a hollow organ of a patient 15. To operate the robot system 2, a remotely arranged operating unit 17 is provided. The remotely arranged operating unit 17 is connected to the robot control unit 8 via a data transmission link 18 (e.g., wireless data transmission link 18). Remotely arranged may be that the operating unit 17 is located at least in a different room from the examination room, but, for example, in another building or even another hospital (e.g., in another town or even country). Such remotely arranged control enables a specialist to perform operations at various locations without having to travel there, so that, effectively, a much higher number of operations is possible.

Additionally, the overall system 1 may have a memory unit 31 for storing various data, image data, and information. The system may also have a communication apparatus (not shown) for retrieving medical data or information from external storage arrangements or databases. In addition, a display unit 18 for displaying image data and other data is assigned to the overall system 1. The display unit 30 may be viewable by the user (e.g., also arranged remotely). Additionally or alternatively, an overall system control unit may also be present.

The fundamental method is shown in FIG. 1. In a first act 21, data is supplied for a planned navigation procedure for an object through a hollow organ with at least one navigation step. The planning of the navigation procedure may have been drawn up, for example, by using a known planning tool. Such planning may be produced, for example, based on 2D or 3D X-ray images (e.g., CT, Angio) or some other imaging method. The data for the planned procedure may be retrieved or supplied either direct from the planning tool, a memory (e.g., memory unit 31), or from a cloud via a further data transmission link. The data may also contain, aside from the property of the data transmission link 18 to be used (e.g., a data transmission rate or bandwidth), the nature of the planned navigation procedure, the number and nature of the navigation steps, and/or a sequence of steps for the navigation procedure, patient data (e.g., weight and/or age and/or height of the patient), and/or data relating to the hollow organ (e.g., structure and/or anatomy of the hollow organ), and/or object data (e.g., which object/device is to be used and also data relating to its shape, size, rigidity, etc.), and/or a medicament to be applied, and/or a contrast agent to be used, and/or device data, and/or X-ray parameters. In this regard, the data is forwarded, for example, to an evaluation system (e.g., evaluation unit 16).

In a second act 22, the supplied data is evaluated in terms of a performability level of the navigation procedure (e.g., by an evaluation system, such as. evaluation unit 16).

A performability level represents a value for the absolute or relative performability of the planned navigation procedure (e.g., whether performance of the procedure is possible or probable without restrictions, with restrictions, or not at all). In this regard, various subdivisions of a performability level may be provided. Thus, the performability level may include a simple subdivision into performable/not performable, a subdivision into multiple stages, or a very precise subdivision (e.g., probability in percent). Additionally, the performability level may be dependent on the corresponding person giving treatment or the device used.

In this regard, the evaluation may be carried out either based on a comparison with empirical data and/or based on a theoretical model and/or by using a learning-based algorithm.

A comparison with empirical data, for example, may refer back to a memory (e.g., memory unit 31) and/or a table (e.g., look-up table) with a large quantity of previously performed and/or collected data, with which the current data is compared. The data may then be dependent on or independent of the person giving treatment, for example. Alternatively or additionally, one or more theoretical model calculations may be carried out to obtain a performability level. A learning-based algorithm (e.g., previously trained with a large quantity of previously performed and/or collected data) may also be used for the determination of the performability level. Also, a dependence on the person giving treatment is possible.

Subsequently, in a third act 23, an evaluation result is output to an output unit (e.g., to a display unit, such as a monitor, tablet, etc.). The nature of output of the evaluation result may embrace a number of different options. Thus, for example, a simple color subdivision may be output (e.g., red for a non-performable and green for a performable procedure), or a further subdivision with more than two colors (e.g., traffic lights: red, orange, green) may be output. Pictorial representations, scalar displays, or text/numbers that describe the performability may also be output. For example, a probability that the procedure is performable may also be output. Further, optical, acoustic, or haptic warnings may also be output if, for example, the evaluation produces the result that performability of the navigation procedure is not provided or at least is provided with low or medium probability. The output enables the person giving treatment to identify rapidly and by simple means whether performability of the procedure is reliably provided, and may initiate corresponding acts where necessary. In addition, a recommendation may also be output (e.g., whether the procedure should be started or not).

As shown in FIG. 2, aside from the output of the evaluation result, in a fourth act 24, a suggestion may be output for a modified (e.g., in at least one parameter) navigation procedure. For this, the evaluation system may likewise determine the performability level for such a modified navigation procedure, and this may be displayed together with the suggested modifications or the suggested modified procedure. In one embodiment, a suggestion for modifying the planned navigation procedure is output, where the navigation procedure adapted by the modification has a better performability level, or a higher or at least the same probability of performability. A modification may relate to, for example, the property of the data transmission link to be used, the nature of the planned navigation procedure, the quantity and nature of the navigation steps, and/or a step sequence of the navigation procedure, the object data (e.g., which object, shape, size, rigidity, etc.), or medicaments to be applied, or contrast agent to be used, or device data or X-ray parameters or other such modifications. Parameters that may not be influenced (e.g., patient data) may not be modified and accordingly are not included in the modification suggestion. In terms of, for example, the properties of the data transmission link, potential alternative data links, alternative times, or other modifications may be displayed. If the property of the data transmission link may not be influenced, then other parameters may be modified, which together with the data transmission link, produce a safer or more harmonious procedure with a better performability level or greater probability of performability. Provision may also be made to output a modification suggestion that has the highest or best possible performability, which the evaluation unit calculates. The navigation procedure may therefore be optimized, for example, in terms of the performability level with reference to the modifiable parameters. As described models, a trained algorithm or an empirical database may be used during the determination of modified navigation procedures and the corresponding performability levels.

Additionally, not only one suggestion but two or more suggestions may be output, where, for example, an indication based on the performability level is also output for each suggestion. This makes it apparent to the person giving treatment which suggestion has the highest performability level or the highest probability of being performed successfully. Provision may then be made, for example, for the person giving treatment to select the suggestion for a navigation procedure that has the highest or at least a high probability of success.

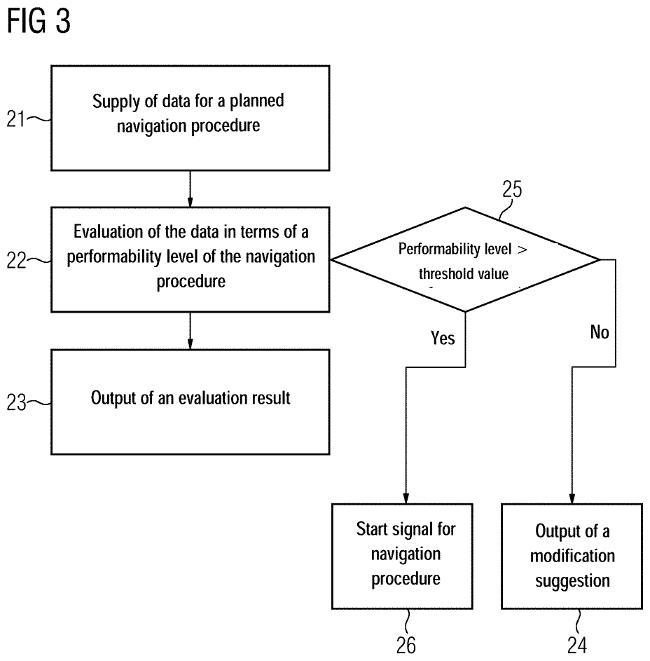

FIG. 3 shows a further method in which a threshold value is brought in for the performability level. The threshold value for the performability level or for the probability that the procedure is performable may be preset or selectable by a user. In the context of the evaluation, the question of whether the performability level (e.g., in the form of a probability) lies above the preset threshold is determined by the evaluation unit in a fifth act 25. If so, then a start signal for an automatic or semi-automatic navigation procedure is subsequently given automatically in a sixth act 26. If not, then one or more modification suggestions, together with navigation procedures having performability levels exceed the threshold value, are output in a fourth act.

Following the method, the evaluation and/or the evaluation result may be deposited or stored in a database or table (e.g., a look-up table) for use in a subsequent method.

As a result of the method, a person giving treatment in invasive operations with a navigation procedure may monitor all relevant steps of the navigation in a simple manner. The person giving treatment may identify from the evaluation result of the evaluation unit whether and/or at which step problems that adversely affect the performability of the procedure may arise, and where to relevantly intervene, alter, or postpone the operation. This is also important in the case of operations that are undertaken by remote access and with the use of robot systems, which carry out various aspects of a procedure (semi-)automatically. Overall, the quality of an operation is enhanced, and safety is substantially improved by the method.

The method incorporates an a-priori analysis of the performability of a planned robot or robot-supported operation based on relevant information about the planned procedure. The procedures may involve, for example, endovascular procedures. In this context, a person giving treatment may be provided with a recommendation. For example, with regard to operations that are undertaken by remote access (e.g., from a remotely arranged user unit) and where a robot system performs various aspects of a procedure (semi-)automatically, the method makes it possible for the connected person giving treatment to monitor all relevant steps and thereby enhance the safety to the overall system.

For the most precise possible analysis of performability, the evaluation system or the evaluation unit may be supplied with, among other things, information about the planned procedure, the patient (e.g., a digital twin of the patient), and about the devices used (e.g., rigidity or purpose of use). Additionally, this information or individual parts may be stored in a database and therefore be used as a standard of comparison for other procedures, or be included or used in their analysis. Further, with regard to a remotely activated navigation procedure, the quality of the data transmission link that is probably available (e.g., the data transmission bandwidth) is also included in the analysis and the subsequent recommendation to the person giving treatment. Particularly in light of potential delay times, additional potential safe treatment options may additionally be displayed.

Last, the recommendation to the person giving treatment may be derived based on mathematical models, statistical findings, or a trained function. The decision of the person giving treatment and an evaluation of the procedure (e.g., a (semi-)automatic evaluation), or of the success of the procedure, may likewise subsequently be deposited in a database or also used to refine the analysis system. Additionally, alternatives, such as in terms of imaging or devices, may be suggested to the person giving treatment so as to raise the probability of safe performability.

A hollow organ 32 of a patient may be, for example, a vessel (e.g., an artery or vein or bronchial tube), a segment of a vascular system, or the whole vascular system of a patient.

For a particularly safe navigation procedure, a method is provided for planning a remote-controlled navigation of medical objects in a hollow organ of a patient. The navigation is capable of being performed by robot or in a robot-supported manner using a robot system, and is visually monitored by an imaging system. The robot system has a drive system, a robot control unit, and at least one input unit arranged at a distance from the robot control unit. At least one data transmission link is present (e.g., between the robot control unit and the input unit). The method includes supplying data for a planned navigation procedure of an object through a hollow organ with at least one navigation step. The supplied data is evaluated by an evaluation system in terms of a performability level of the navigation procedure. The evaluation is carried out based on a comparison with empirical data and/or based on a theoretical model, and/or using a learning-based algorithm. An evaluation result is output to an output unit.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for planning a remote-controlled navigation of medical objects in a hollow organ of a patient, the navigation being performable by robot or in a robot-supported manner using a robot system, and being visually monitored using an imaging system, wherein the robot system includes a drive system, a robot control unit, and at least one input unit arranged at a distance from the robot control unit, and wherein at least one data transmission link is present, the method comprising:
supplying data for a planned navigation procedure of an object through the hollow organ with at least one navigation step;
evaluating, by an evaluation system, the supplied data in terms of a performability level of the planned navigation procedure, wherein the evaluating is carried out based on a comparison with empirical data, based on a theoretical model, using a learning-based algorithm, or any combination thereof; and
outputting an evaluation result to an output unit.

2. The method of claim 1, wherein the output evaluation result includes a probability, with which the navigation procedure is performable.

3. The method of claim 1, wherein the supplied data includes at least one property of the data transmission link to be used, and
wherein the evaluating takes account of at least the at least one property of the data transmission link.

4. The method of claim 3, wherein the at least one property of the data transmission link includes a data transmission rate.

5. The method of claim 3, wherein the supplied data also includes a type of the planned navigation procedure, a sequence of steps in the planned navigation procedure, patient data, data for the hollow organ, object data, a medicament to be applied, a contrast agent to be used, device data, X-ray parameters, user-specific data, or any combination thereof.

6. The method of claim 2, wherein the output evaluation result includes at least one suggestion for modifying the planned navigation procedure, and
wherein a navigation procedure adapted by the modification has a higher or at least the same probability of performability compared to the planned navigation procedure.

7. The method of claim 1, wherein the output evaluation result includes multiple suggestions including navigation procedures differing from the planned navigation procedure with a statement based on respective levels of performability.

8. The method of claim 6, wherein the at least one modification suggestion has modifications with regard to at least one navigation step, with regard to the object used, with regard to the planned navigation path, with regard to a contrast agent, with regard to a medicament, with regard to X-ray parameters, or with regard to any combination thereof.

9. The method of claim 1, wherein the evaluation, the evaluation result, or the evaluation and the evaluation result are deposited in a database or table for use in a subsequent method.

10. The method of claim 1, wherein a starting signal for the planned navigation procedure is automatically triggered when the performability level reaches or exceeds a preset threshold value.

11. The method of claim 1, wherein the at least one data transmission link includes a data transmission link between the robot control unit and the input unit.

12. A system comprising:
a robot system comprising:
at least one robot control unit;
a robot-supported drive system; and
an input unit arranged at a distance from the at least one robot control unit, the at least one robot control unit being configured to activate a robot-supported navigation of a medical object in a hollow organ of a patient using the robot-supported drive system, wherein at least one data transmission link is present between the at least one robot control unit and the input unit;
an imaging system for visual monitoring of the robot-supported navigation, the imaging system comprising:
a beam source and an image detector for recording projection images; and
a system control unit configured to activate the imaging system;
an evaluation unit configured to evaluate supplied navigation planning data with regard to a performability level of a navigation procedure; and
an output unit configured to output an evaluation result,
wherein the evaluation unit is configured to carry out the evaluation based on a comparison with empirical data, based on a theoretical model, using a learning-based method, or any combination thereof.

13. The system of claim 12, wherein the imaging system includes an X-ray system.

14. The system of claim 12, wherein the supplied data includes at least one property of the data transmission link, and
wherein at least the property of the data transmission link is taken into account in the evaluation.

15. The system of claim 12, wherein the output evaluation result includes a probability, with which the robot-supported navigation procedure is performable.

16. The system of claim 12, wherein the supplied navigation planning data includes at least one property of the data transmission link to be used, and
wherein the evaluation takes account of at least the at least one property of the data transmission link.

17. The system of claim 16, wherein the at least one property of the data transmission link includes a data transmission rate.

18. The system of claim 16, wherein the supplied navigation planning data also includes a type of the planned navigation procedure, a sequence of steps in the planned navigation procedure, patient data, data for the hollow organ, object data, a medicament to be applied, a contrast agent to be used, device data, X-ray parameters, user-specific data, or any combination thereof.
